(19) United States
(12) Patent Application Publication
(10) Pub. No.: US 2020/0309757 A1
(43) Pub. Date: Oct. 1, 2020

(54) CHEMICAL DETECTION USING A SENSOR ENVIRONMENT

(71) Applicant: International Business Machines Corporation, Armonk, NY (US)

(72) Inventors: Igor S. Ramos, Round Rock, TX (US); Devon E. Mensching, Austin, TX (US); Kimberly J. Taft, Austin, TX (US)

(21) Appl. No.: 16/363,738

(22) Filed: Mar. 25, 2019

(51) Int. Cl. G01N 33/02; G01N 33/00; G01N 31/22; G06Q 30/06; G06T 19/00; G06F 1/16

(52) U.S. Cl. CPC G01N 33/02; G01N 33/0027; G06F 1/163; G06Q 30/0641; G06T 19/006; G01N 31/22

(57) ABSTRACT

A computer receives one or more user preferences regarding a product characteristic. The computer performs, using one or more sensors, product testing of a product to gather characteristic data of the product. The computer determines, using the characteristic data of the product, a measured characteristic value of the product. The computer compares the measured characteristic value with an expected characteristic value associated with the one or more user preferences. The computer determines, based on the comparison, a product suitability value for use by a user. The computer outputs, using the product suitability value, an indication whether the product is suitable for use by the user in accordance with the one or more user preferences.

### BACKGROUND

The detection of certain smells can indicate the presence of harmful substances or the presence of desirable substances.

The freshness of food or characteristics of other types of items can be important for a wide variety of applications, including personal shopping, grocery store managing, food distribution, food delivery, and food inspection. Current methods for determining whether food is fresh, including whether it is not yet ripe, already ripe, or expired, are often non-technical. Methods include visual inspection of products for color, spots, or other indications of freshness, manually grasping products to determine firmness or moisture content, and/or smelling products with a human nose. Often, these existing methods require a level of knowledge on the part of the person inspecting the products to determine what the color, firmness, smell, etc. of a product means regarding the current and/or future freshness of the product.

### SUMMARY

Disclosed herein are embodiments of a method, system, and computer program product for determining product characteristics. A computer receives one or more user preferences regarding a product characteristic. The computer performs, using one or more sensors, product testing of a product to gather characteristic data of the product. The computer determines, using the characteristic data of the product, a measured characteristic value of the product. The computer compares the measured characteristic value with an expected characteristic value associated with the one or more user preferences. The computer determines, based on the comparison, a product suitability value for use by a user. The computer outputs, using the product suitability value, an indication whether the product is suitable for use by the user in accordance with the one or more user preferences.

The above summary is not intended to describe each illustrated embodiment or every implementation of the present disclosure.

### BRIEF DESCRIPTION OF THE DRAWINGS

The drawings included in the present application are incorporated into, and form part of, the specification. They illustrate embodiments of the present disclosure and, along with the description, serve to explain the principles of the disclosure. The drawings are only illustrative of certain embodiments and do not limit the disclosure.

FIG. 1 depicts a block diagram of a network connected sensor environment, in accordance with embodiments of the present disclosure.

FIG. 2 depicts an example method for determining product characteristics using a sensor environment, in accordance with embodiments of the present disclosure.

FIG. 3 depicts an example method for chemical detection and visual display using a sensor environment, in accordance with embodiments of the present disclosure.

FIG. 4 illustrates a block diagram of a computer system, in accordance with some embodiments of the present disclosure.

While the present disclosure is amenable to various modifications and alternative forms, specifics thereof have been shown by way of example in the drawings and will be described in detail. It should be understood, however, that the intention is not to limit the present disclosure to the particular embodiments described. On the contrary, the intention is to cover all modifications, equivalents, and alternatives falling within the spirit and scope of the present disclosure.

### DETAILED DESCRIPTION

Embodiments of the present disclosure recognize that the human olfactory system is limited in its ability to detect and differentiate between various odors. In addition, embodiments of the present disclosure recognize that smells may be interpreted differently by different people. Embodiments of the present disclosure recognize that locating the source of a given odor can present challenges that are not easily overcome without significant time and effort. Embodiments of the present disclosure provide a system that presents an identified odor as visual information, i.e., the identified chemicals that comprise and odor are shown using one or more of shapes and/or colors on a visual display.

Some embodiments of the present disclosure provide for a method, system, and computer program product for detecting chemical odors using a sensor environment. This sensor environment can include augmented reality, such as augmented reality glasses or goggles with cameras, which can detect light in a variety spectrums, including those outside of the human range such as ultraviolet and infrared, a glove that can include a tactile sensor and/or a set of chemical sensors that can detect the presence of chemical in a sample that is in contact with the glove and may include an olfactory sensor to detect chemicals that are in a gaseous state in the air, and additional sensors as appropriate, and a computer to process the received input. Using user preferences and configurations, the sensor input can perform testing on various samples, including the air and objects in an environment, to determine the source of a given chemical.

In one embodiment and scenario, an employee in a hospital can note a scent of a cleaning agent that seems to be unusually strong. The employee can activate the system, which can include selection of cleaning agents as a category of chemicals to be detected. In other embodiments, selection of a category may be optional. The system gathers data from the environment and matches that data against the chemical signatures of known cleaning agents. The system identifies the most likely cleaning agent based on both the matching as well as the strength of the signal corresponding to that cleaning agent. In this example, the system identified six cleaning agents but one of those six, cleaning agent “A” had a signal that was ten times higher than the others, i.e., the concentration of chemicals in the air that correspond to cleaning agent “A” are ten times higher than the concentrations of chemicals corresponding to the other five cleaning agents. The system can access a profile for cleaning agent “A” and determine that this cleaning agent is visible using the ultraviolet light spectrum. The system can include augmented reality glasses or goggles and the system can configure the video camera on the glasses or goggles to detect the frequency of ultraviolet light and converts that frequency to a frequency that the employee can see. The system also uses the concentration in the air of cleaning agent “A” to generate a range of concentration for tracking the source, e.g., the system tracks places where the concentration is at least ninety percent of the current concentration.

In some embodiments, the employee can activate a “search” mode in the system which configures the system to track a chemical trail. The user can use one or more chemical sensors, including sensors located on a glove, and slowly pass the sensors through the air, as well as over and around various objects and areas. The system can collect a series of measurements and provide a visual indication that conveys the changes in strength of the chemical signature as well as indicating a couple of spots one the floor that are emitting the frequency of ultra violet light, i.e., it is likely that the spots on the floor are droplets of the cleaning agent, so the system highlights those areas in the display. Further the system keeps track of where the chemical signal seems to be the strongest and conveys that information to the user. In some embodiments, the series of measurements may indicate an increase in chemicals along a path and provide an indicator such as an arrow in the direction of increasing level of chemical. As such, the employee is presented with information indicating that the chemical signature is strongest near a closed hallway door. The employee opens the door and proceeds down the hallway passing various rooms. Throughout this process, the system can continue collecting data and providing visual indicators to the employee. The system identifies and indicates several more droplets on the floor. Halfway down the hall, the system indicates a decrease in the chemical signal and indicates that a room that the employee just passed was that last location that had a concentration within the range. The employee enters the room and the concentration of chemical signal increases to five times the initial concentration. There are is also a large spot on the floor under the cart that is emitting the frequency of ultraviolet light. The employee passes the glove over a cart of cleaning supplies and the system indicates that a bottle on the cart is a likely source of the chemical odor based on a significant increase in concentration around the bottle. The employee picks up the bottle and discovers that the bottle has a cracked bottom and has been leaking cleaning agent “A” as the cart had been wheeled down the hall.

Other aspects of certain embodiments of the present disclosure relate generally to determining product characteristics, and more specifically, to determining product freshness using a sensor environment. While the present disclosure is not necessarily limited to such applications, various aspects of the disclosure can be appreciated through a discussion of various examples using this context.

Various entities are interested in the freshness of food or other products. Users performing personal shopping can be interested in whether any given product, including produce such as bananas, avocados, tomatoes, etc., are ripe for consumption or will be ripe for consumption at a desired date in the future. Grocery store owners or managers can be interested in placing products in a manner such that those which will be unable to be sold for very much longer due to freshness concerns are placed where they will be more likely to be purchased and/or are labeled appropriately such that consumers know when they will best be used by. Distributors or grocery delivery service companies may similarly be interested in which products must be sold or delivered sooner than others to meet consumer demands and provide a high-quality service to customers. Food service inspectors, such as those investigating a restaurant for quality can also be interested in whether products are spoiled and unfit for human consumption or if they do not meet food safety requirements. Chemical supply companies can be interested in the freshness of their chemicals which can degrade over time, particularly unstable chemicals.

Currently, such people and companies can use human senses and knowledge about product characteristics that change with the freshness of the product to determine a current freshness level of a given item. For example, many food products vary in color depending on the freshness of the product, including bananas which can range from green (not yet ripe), to yellow (ripe), to brown or black (over-ripe or spoiled)), and visual inspection of such food products can provide information regarding their freshness. Along with color variations, some food products develop spots or other changes in the visual consistency of the product as they ripen or pass their optimal freshness (e.g., spots on bananas or avocados). More generally, chemical compounds (including those present in foods) can change color over time due to ongoing processes or reactions, such as oxidation (e.g., a metal rusting) or exposure to chemicals in the air, heat sources, or other causes.

Food products may also vary in firmness based on freshness of the product. Various fruits and vegetables can be firm before they are ripe and soften over time, eventually becoming too soft or “mushy,” with the preferred time to eat such food products being when they are at an intermediate point between firm and mushy. Touching, holding, and/or grasping such food products can provide information regarding their freshness. In addition to firmness, other aspects of food products may be examined by touch, including finding lumps, bumpiness, or abnormal formations on or below the surface of a food product. Chemical change, including degradation over time, can result in a changed melting or boiling point of a chemical which can result in a source of the chemical becoming softer or harder as it degrades over time, which can be detected with a tactile sensor.

Many fruits have differing odors depending on their freshness or ripeness and smelling such food products or the air surrounding them can provide information regarding their freshness. For example, strawberries and melons which smell sweet, but not to an excessive degree, can be at or near their optimal freshness. On the other hand, a smell of mold, decay, fermentation, or otherwise objectionable smell can be present in a variety of food substances and indicate that they are not fresh. Some such smells may be the result of, or indicative of, bacterial growth, which may be more accurately measured with a sensor designed to measure bacteria. Many chemicals or other products beyond those present in food items can emit an odor, which is generally indicative of a quantity of the chemical (or a byproduct of a given chemical reaction) being present in the gaseous phase. This can occur especially with a liquid, where there can be an equilibrium between an amount of the chemical in liquid form and an amount of the chemical in gaseous form but can also occur with a solid due to a process of sublimation that releases the chemical in a gaseous form. One having ordinary skill in the art can appreciate that many materials release chemicals (i) in gaseous form or (ii) in a liquid form and that these chemicals can undergo a number of physical changes and/or chemical reactions that release various gaseous products.

Because the freshness of food and other products is typically observed using human senses, experience and knowledge of what to observe and sense can play an important role in determining freshness accurately. A first-time avocado shopper may not know what to purchase without assistance. Shopping for food products in a foreign location with different types of produce can add an additional challenge. Purchasing a new type of food product for placement in a grocery store can similarly present a store manager with a challenge.

Some embodiments of the present disclosure provide for a method, system, and computer program product for determining product freshness using a sensor environment. This sensor environment can include augmented reality, such as augmented reality glasses or goggles, which can receive visual input useful for determining colors and spots of food and other products, a tactile sensor useful for determining the firmness of food and other products, an olfactory sensor to detect smells of food and other products, a bacterial sensor for determining bacterial levels of food and other products, additional sensors as appropriate, and a computer to process the received input. Using user preferences and configurations, the sensor input can perform product testing on food or other products, determine product freshness, compare product freshness with a desired use for the product, and determine whether a food or other product is suitable for its intended use. This process can be improved by receiving user feedback to train the system to more accurately identify product freshness.

In some embodiments, a food distribution system can leverage the present disclosure to fulfill customer orders. In such embodiments, various foods can have different ages and/or stages of ripeness. For example, a banana can range from green to very yellow with lots of brown spots to completely brownish-black. As such, when users provide their order of produce, they also select an age and/or ripeness aspect. For other products such as, for example, meats, a different characteristic may be used such as fat content or marbling of the meat. The system leverages the visual data as well as the chemical data to identify which products meet these requirements. In this way, the customer receives products that are much more likely to match what the customer desired. In some such embodiments, a training session is leveraged in which the user is presented with images of products with various degrees of a given characteristic and based on the user's selections, the system defines a range for that characteristic. For example, a range indicates the most ripeness and least ripeness a customer desires for a given fruit.

A sensor environment and process as described herein provide advantages over prior methods of determining food or other product freshness, by enabling automation, eliminating variation based upon human senses between different people, and streamlining food and other product freshness determination. Additionally, the use of a sensor environment can assist a user who may be unable to determine freshness without the aid of technology due to inexperience or physical limitation (e.g., blindness or color blindness may limit a user when the color of the food or other product indicates freshness or anosmia (loss of sense of smell) may limit a user when the smell of a food or other product indicates freshness). These improvements and/or advantages are a non-exhaustive list of example advantages. Embodiments of the present disclosure exist which can contain none, some, or all of the aforementioned advantages and/or improvements.

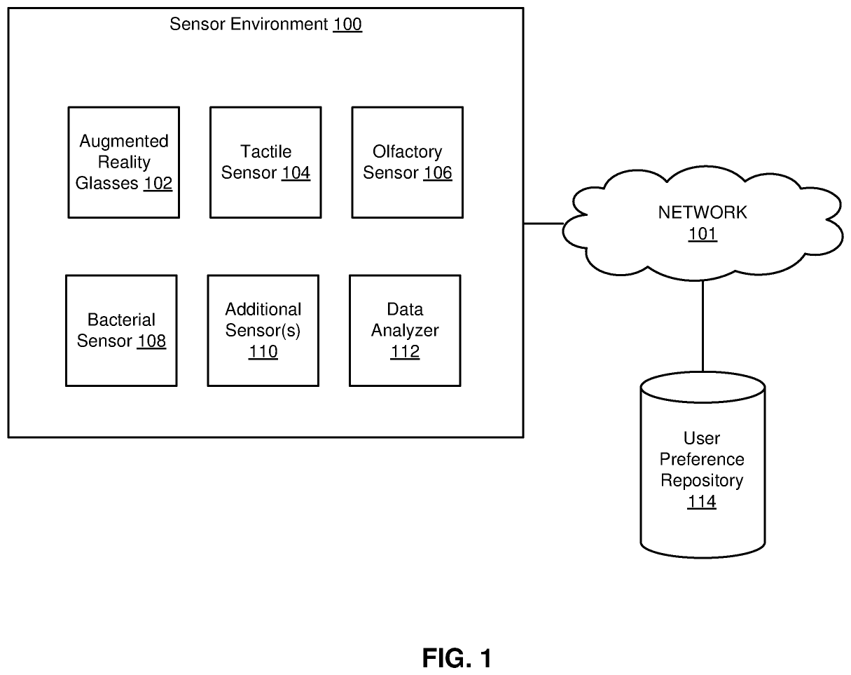

Referring now to FIG. 1, depicted is a block diagram of a network-connected augmented reality environment, in accordance with embodiments of the present disclosure. FIG. 1 shows a sensor environment 100 which includes a network 101, augmented reality glasses 102, tactile sensor 104, olfactory sensor 106, bacterial sensor 108, additional sensor(s) 110, and data analyzer 112. Sensor environment 100 can be more or less complicated in embodiments, including additional sensors, components, computers, or the like. Additionally, one or more components may be combined or omitted in embodiments.

Augmented reality glasses 102 can be present as part of sensor environment 100 and aid a user in multiple ways. As used herein, augmented reality glasses include all forms of augmented reality eyewear such as glasses, goggles, contact lenses, headset, etc. A user wearing augmented reality glasses 102 can focus their vision on (and in doing so aim the glasses at) one or more food or other products that they wish to determine the freshness of and the augmented reality glasses can receive visual input of the color, shape, and/or any patterns such as spotting of the food or other products. In some embodiments, a user can aim augmented reality glasses 102 at an environment for detection of one or more chemicals or other visual input data useful for chemical sensing or testing. In some embodiments, the augmented reality glasses 102 can also provide results to the user. This may take the form of text pop-up messages in a display of the glasses 102. The messages can be located near where a food or other product is visible through the glasses 102. The messages can indicate information regarding the freshness of the food or other product, such as, but not limited to, indicating it is fresh currently, will be most fresh in a specified time period (e.g., 2 days from now), or that it is not fresh (which may be specified as “not ripe,” “spoiled,” or another indicator that it is not fresh).

In some embodiments, the augmented reality glasses 102 can highlight one or more food or other products visible in a display of the augmented reality glasses 102. For example, if a user is viewing a display shelf of pears containing a plurality of pears, the augmented reality glasses 102 can receive visual input of the pears, the sensor environment 100 (or a portion thereof) can perform a determination of freshness for each of the pears, and the augmented reality glasses can highlight pears which are currently fresh in one color or pattern; highlight pears which are not yet ripe, but will be ready for consumption in 3 days, in a second color or pattern; and highlight pears which are no longer fresh in a third color or pattern. Many variations on this concept will be apparent to those of skill in the art, including more or less than three colors/patterns, time periods other than 3 days, use with other products, etc.

In some embodiments, a user may use the displayed highlights from augmented reality glasses 102 to sort the food or other products into different display shelves, bins, or otherwise arrange the food or other products in a manner consistent with business needs. In another example, food products may be placed together in a package such that some of the food products are currently fresh and others will be fresh in the near future such that a user who buys the package will be able to enjoy the products over time and neither have to wait for them all to be ripe, nor consume/serve them all immediately.

The augmented reality glasses 102 can also display a visual indication of the output of one or more of the other sensors of sensor environment 100. For example, augmented reality glasses 102 can display an output from olfactory sensor 106 by highlighting a region where chemicals were sensed by olfactory sensor. This display can vary in intensity or otherwise provide an indication based on the amount of odor or chemical detected at a plurality of detection points. This display can also include one or more indicators (such as an arrow) marking a direction of an increase in intensity.

In some embodiments, augmented reality glasses 102 can display prompts for additional input, such as an instruction to use a tactile glove or other sensor on an identified food product such that sensor environment 100 can receive additional input for determining freshness of the product.

In some embodiments, augmented reality glasses 102 can be replaced with a video camera or still photo camera for receiving visual input. In such embodiments, in addition to the camera, a display or screen (which may be part of the camera or may be separate) may be used to provide information to the user such as the above described highlighting, prompts, or output of other sensors. For example, in some embodiments, a user may be able to use a cell phone's camera to capture video or still images of the same display shelf of pears in the example above. Continuing with the example, the sensor environment 100 can perform the freshness determination and provide an overlay of highlighting on one or more images of the display shelf on the cell phone's screen or otherwise provide prompts or information to the user on the screen.

Tactile sensor 104 can take various forms in various embodiments. Tactile sensor 104 can be a tactile glove, which can measure the force applied by a hand within the glove and the resistance of a food product being gripped to determine the firmness of the food or other product. This firmness data can be used in determining freshness of products which change in firmness based upon freshness (e.g., a hard avocado may indicate it is not yet ripe compared to a soft avocado which is ripe compared to a softer avocado which is past ripe). In some embodiments, instead of a tactile glove, tactile sensor 104 can be a penetrometer used to determine firmness of a product; however, use of a penetrometer is generally destructive (e.g., a product may need to be sliced open to use it) and may not be suitable for all uses. A tactile sensor 104 in the form of a penetrometer may be useful for testing a sample of a larger quantity of food products (e.g., one apple out of a bushel), where destruction of the sample is acceptable to gain information about the larger quantity.

In other embodiments, tactile sensor 104 can be a robotic hand or other automatic grasping device which may be used to apply pressure to a food or other product and measure the response of the food product. In an embodiment using a tactile sensor 104 in the form of a robotic hand or other automatic grasping device, tactile sensor 104 may be programmable or adjustable to apply varying levels of pressure depending on the food product to be tested with the tactile sensor. An automated form of tactile sensor 104 may be preferable for applications where many food products are to be tested and may be less desirable for individual shoppers.

In some embodiments, a tactile sensor 104 in the form of a glove can include one or more of the other sensors of sensor environment 100. For example, olfactory sensor 106 and/or bacterial sensor 108 can be located on the same glove as tactile sensor 104. In some embodiments, a user can select a mode for the glove such that it can be used for detection or receipt of sensor input regarding one of touch, smell, or bacteria, while in other embodiments, such a glove can receive sensor input for a plurality of types of sensors located on the glove at once.

Olfactory sensor 106 can employ various machine olfaction techniques to simulate the sensation of smell. Olfactory sensor 106 can intake a sample of air and analyze the sample to determine the chemical makeup of the air sample. Depending on the nature of olfactory sensor 106, this can include placing the food or other product within a container and detecting whether any gases are being emitted from the food or other product, placing the sensor near the food or other product and analyzing the air near the product, or any other means for collecting an air sample relating to the food or other product. In some embodiments, olfactory sensor 106 can be located on a glove, such as tactile sensor 104, and a user can place their hand wearing the glove on or near a product or area to be tested. In some embodiments, this can include targeted detection of one or more gases which relate to a particular food or other product's freshness, such as detection of gases which emanate from mold or bacteria. In some embodiments, this can entail creating a scent profile for a food or other product based on one or more chemicals being emitted by the food or other product. In some embodiments, olfactory sensor 106 may be trained by providing control samples of air from food or other products of known freshness levels such that comparison of test samples can be performed relative to the controls. Olfactory sensor 106 may use various types of sensor technology including conductive-polymer odor sensors (such as polypyrrole), tin-oxide gas sensors, and quartz-crystal micro-balance sensors. Olfactory sensor 106 may also use various types of chemical differentiation techniques including gas chromatography. Olfactory sensor 106 can include one or more sensor components that change color when exposed to a particular gas.

Bacterial sensor 108 can take various forms in embodiments and may depend on one or more types of bacteria to be detected. In some embodiments, bacterial sensor 108 can be an array of a plurality of sensors with each sensor configured to detect one or more specific types of bacteria. Various types of bacterial sensors exist in the art and can be used as a part of sensor environment 100. Possible types of bacterial sensors include sensors with one or more chemicals designed to bind to bacteria and produce a detectable response upon binding. For use in sensor environment 100, bacterial sensor 108 may target specific food-borne bacterial such as E. coli (Escherichia coli), Salmonella, etc.

Additional sensor(s) 110 can include additional sensors of the same types as discussed above to operate as fail-safes or backup sensors or can be other types of sensors which can detect one or more properties of a food product which could be used in determining the freshness of the food product.

Data analyzer 112 can be a computer configured to receive the input from one or more of augmented reality glasses 102, tactile sensor 104, olfactory sensor 106, bacterial sensor 108, and additional sensor(s) 110. Data analyzer 112 can use this received input and compare with a database and/or use algorithms to determine based upon the received input whether the food or other product(s) being sensed are fresh. In some embodiments, this can include a determination of when the food or other product(s) will be at their peak or optimal freshness and/or when they will spoil or no longer be fresh. Data analyzer 112 can aggregate the received input from more than one sensor when appropriate to increase the confidence level of a freshness determination. Data analyzer 112 can perform tasks such as identifying one or more chemicals based on sensor data received from one or more of the sensors in sensor environment 100, which may involve comparing the sensor data to reference data. Data analyzer 112 may perform additional functions as part of sensor environment 100 including providing display information or other output which is available to a user of sensor environment 100 and/or prompts for a user to gather additional data to analyze. Data analyzer 112 is depicted in FIG. 1 as part of sensor environment 100, but in other embodiments, data analyzer 112 may be a separate computer, may be a virtual machine such as in a cloud computing environment, may otherwise be located separately from the sensor environment, or some combination thereof.

Sensor environment 100 is depicted as connected to network 101. This connection can take various forms in embodiments, including a physical connection, such as by ethernet cable, or a wireless connection. Network 101 can be the internet, a local area network (LAN), a company intranet, a combination of networks, or other network configuration. Also connected to network 101 is user preference repository 114. User preference repository 114 can be used to store user preferences regarding the use of sensor environment 100, product freshness preferences, and/or user recipes. The information stored in user repository 114 can vary in embodiments. User preference repository 114 can contain the preferences of one user, can be an aggregation of user preferences, or can store separate preferences for multiple users. In some embodiments, sensor environment will not be network connected and user preferences can be stored within sensor environment 100 within one or more of its components.

In a similar fashion, one or more other repositories of information (not shown) can exist and either be present within sensor environment 100 or connected to sensor environment 100, such as by network 101, which sensor environment 100 can access for control samples, reference data, or other information useful in interpreting the data collected by sensor environment 100, and may be used by data analyzer 112 in performing data analysis.

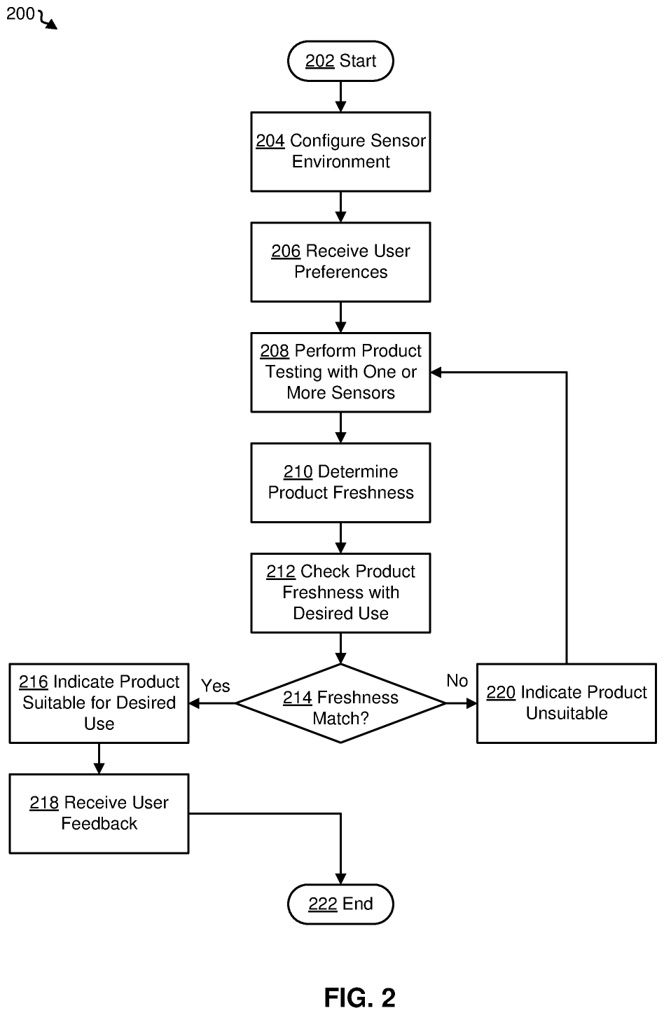

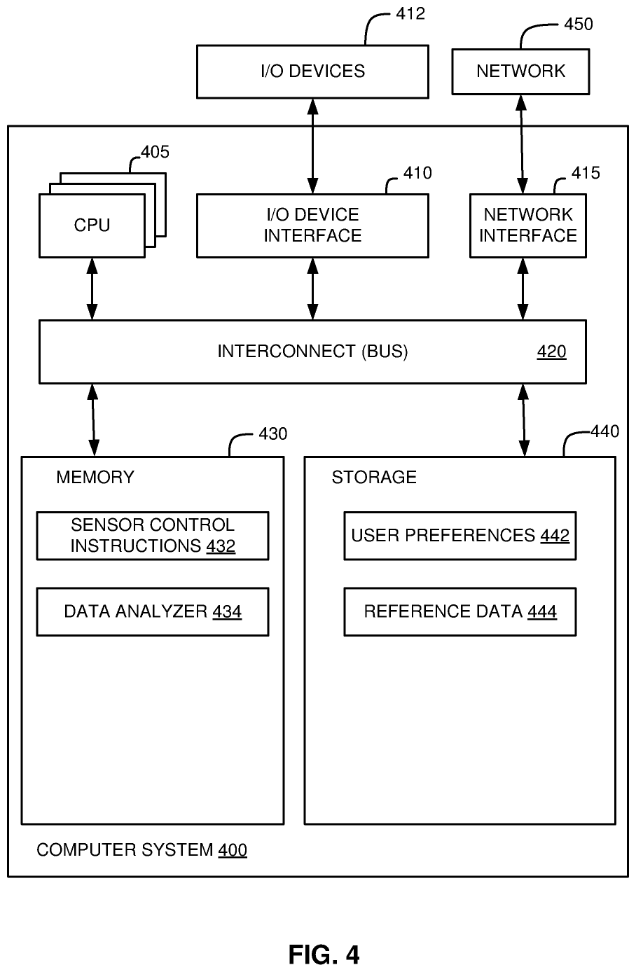

Referring now to FIG. 2, depicted is an example method 200 for determining product characteristics (in this embodiment freshness) using a sensor environment, in accordance with embodiments of the present disclosure. Method 200 can include more or fewer operations than those depicted. Method 200 can include operations in different orders than those depicted. In some embodiments, method 200 can be performed by or performed using a sensor environment (such as sensor environment 100 depicted in FIG. 1) and/or by a computer system (such as computer system 400 depicted in FIG. 4).

From start 202, method 200 proceeds to 204 to configure the sensor environment. Configuration of the sensor environment will vary in embodiments. In some embodiments, configuration of the sensor environment will include turning on augmented reality glasses and/or activating a food or other product freshness application or setting for such glasses, cell phone, or other device. Configuration of the sensor environment can include connecting and/or activating one or more sensors including augmented reality glasses, a tactile sensor, an olfactory sensor, a bacterial sensor, and/or any additional types of sensors as applicable. Various types of sensors may be useful for certain applications and may be unnecessary for other applications. For example, determining the freshness of a fruit or vegetable may utilize a tactile sensor whereas determining the freshness of meat may utilize a bacterial sensor. Configuration of the sensor environment may also include activating a computer connected to one or more of the sensors. Configuration of the sensor environment at 204 can also include selecting a mode of operation for the sensor environment in embodiments where more than one mode exists, such as, but not limited to, a food freshness mode, a chemical freshness mode, and/or a chemical sensing mode.

At 206, the sensor environment receives user preferences. In some embodiments, a user may manually input preferences into the sensor environment, such as by entering data through a computer interface or through a mobile device. The types of user preferences can depend on the use of the sensor environment. For example, if the sensor environment is being used by a shopper selecting products for purchase and consumption, the input of user preferences can include entering a shopping list, one or more dates for which products are to be used, or other preferences. For example, if a shopper wishes to make guacamole on the upcoming Saturday evening, but is shopping on Wednesday, they can input that information such that the sensor environment will be used to determine which avocadoes will most likely be ripe three days in the future (on Saturday), as opposed to which avocadoes are ripe on the day of shopping. Similarly, if bananas are to be used for baking into banana bread rather than for uncooked consumption, bananas which are further along in ripeness may be acceptable or preferable to bananas which are meant to be consumed without cooking. In some embodiments, user preferences such as shopping lists or advanced meal plans may be detected from social media posts, calendar invites, email messages, or other information available to the sensor environment. In some embodiments, this may occur by a user linking one or more accounts with the sensor environment or importing data from one or more such sources.

In a different example, if the sensor environment is being used by a grocery store manager, they can input preferences that rather than selecting one or more products for purchase, all products can be evaluated for relative freshness, such that products can be sorted into one or more bins or labeled appropriately. A grocery delivery service may utilize a similar set of preferences as an individual user would, but on a larger scale such that various food products can be selected for a plurality of users based on their purchase information, any inputted preferences as to freshness of food products to be received, and/or delivery time to reach such customers. If the sensor environment is to be used in restaurant inspection, the user preferences may include a rating scale such that food products inspected are given a rating in stars, on a scale from 1 to 10, or various other means for ranking the restaurant's food product freshness. Various additional types of user preferences can be used in various embodiments.

In another example, a chemical supply company can input preferences regarding relative purity levels of chemicals. For example, a chemical supply company may supply a chemical at purity levels of 99.7% pure (reagent grade), 90% pure (laboratory grade), and 50% (technical grade). These purity levels, grade labels, or other such settings may be received by the system at 206, such that in the later operations of method 200 are performed including when product testing is performed, the chemicals can be sorted into their respective purity levels and/or determined to be suitable or unsuitable for the purity level they are labeled at. These example purity levels and grade names are provided for example purposes only. Many such levels and grades can exist in embodiments.

At 208, the sensor environment can perform product testing with one or more sensors. The nature of the testing will depend on which sensors are involved in the sensor environment, the product(s) being tested, and the user preferences. For example, in an embodiment where augmented reality glasses are being used, product testing can include the user viewing one or more products such that the augmented reality glasses can receive visual input data. This visual input data can be used in determining freshness of products which change color based upon their freshness. In some embodiments, rather than augmented reality glasses, visual input may be collected via video camera or still photos instead.

In an embodiment where a tactile sensor is being used, a user can manipulate the tactile sensor and a product to be tested to collect the appropriate input. For example, a tactile glove may be used by placing it over a user's hand, which is then used to squeeze a product for testing. The tactile glove can measure the force applied and the resistance of the product to determine its firmness. This firmness data can be used in determining freshness of products which change in firmness based upon freshness (e.g., a hard apple may indicate freshness compared to a soft or “mushy” apple which is no longer fresh).

In embodiments employing an olfactory sensor, a user can place the olfactory sensor near or on one or more food or other products to be tested or place the food or other products within a receptacle connected to the olfactory sensor for testing. This can be used to simulate the detection of smell by detecting the composition of the air surrounding the food or other product.

In an embodiment where a bacterial sensor is used, the bacterial sensor may be touched to a surface of a food or other product or in some embodiments, may have a probe or other appendage inserted into a food or other product to detect the presence of bacteria. This can include detection for harmful bacteria such as E. coli and Salmonella.

In some embodiments, rather than testing with multiple sensors at once at operation 208, the sensor environment can proceed to 210 to determine product freshness upon receiving the first sensor input and can return to operation 208 if additional data is required to make a determination.

At 210, the sensor environment determines the product freshness of one or more products being tested. This can be performed by a data analyzer such as data analyzer 112 of FIG. 1. Determination of product freshness can include comparing the data received from the one or more sensors at 208 with reference samples or other known indicators of freshness or lack thereof. Determination of product freshness at 210 can be a determination of current freshness of the food or other product(s), a future estimated freshness of a food or other product, an expiration date of a food or other product, or some combination thereof. In some embodiments, determination of product freshness can include a rating, such as a numerical indication of freshness. In some embodiments, the determination of product freshness can be a relative determination. For example, a shopper may wish to buy an orange and may want to purchase the freshest orange available at a store, regardless of whether it is ultimately determined to be “fresh” or have a particular freshness rating.

At 212, the sensor environment can check the product freshness determined at 210 with a desired use of the product using the user preferences received at 206. In some embodiments, this can include determining an expected freshness value of a food or other product which would match the desired use of the product according to the user's preferences and comparing the expected value with a value determined in operation 210. In embodiments where a user is shopping for groceries, this can involve checking the determined freshness (which includes an estimated future level of freshness) for the product with the planned future date of using the product. Using the above example of purchasing avocadoes for guacamole to be prepared three days in the future, the sensor environment can check whether the avocadoes will be fresh three days in the future. In other embodiments, this can be checking whether the food product(s) are currently fresh. In embodiments where a grocery store manager (or employee of a grocery store) is checking product freshness, this can include determining if the food product(s) match a bin or display area which advertises that the food products are best by a certain date.

At 214, the sensor environment determines the result of 212 and method 200 proceeds either to 216 to indicate the product is suitable for the desired use or 220 to indicate the product is unsuitable. In some embodiments, rather than a binary determination of suitable or unsuitable, the sensor environment can determine a suitability value which indicates a relative suitability of a product. For example, a suitability value of 10 could indicate that a food product closely matches a desired use and method 200 would proceed to operation 216 as above, whereas a suitability value of 1 could indicate a food product is very unsuitable (e.g., is already spoiled or will most likely be spoiled before the time of intended use) and method 200 would proceed to operation 220 as above. In such embodiments, a threshold value (e.g., a suitability value of 5) could be used where a suitability value above the threshold would count as suitable and any value below would be unsuitable. In some embodiments, the threshold value could be determined by the user preferences received at 206.

At 216, the sensor environment indicates the product is suitable for the desired use based on a freshness match at 214. This can include presenting information to a user of the sensor environment which will vary in embodiments. In some embodiments, the sensor environment can provide information to a user via display on augmented reality glasses, a cell phone screen, or other display. This information can include highlighting regions of a display shelf which contain one or more fresh products, providing a popup message indicating that the product is fresh (which can include more detail such as a freshness rating), and can provide information that it is or will be most fresh on the date of the desired use for the product.

At 218, the sensor environment can receive user feedback. This operation can be optional in some embodiments. A user may be able to provide an indication of agreement or disagreement with the sensor environment to help train the sensor environment such that it can use machine learning to provide better indications of freshness in future instances of method 200. In some embodiments, a user may be able to input a freshness rating, or other rating of the performance of the sensor environment for more precise feedback. In some embodiments, if a user disagrees with the sensor environment's determination that a food or other product is suitable for the desired use, method 200 can return to operation 208 to perform additional product testing.

If at 214, the sensor environment determined there was not a freshness match, the sensor environment proceeds to 220 to provide an indication that the product is unsuitable. This can include presenting information to a user of the sensor environment which will vary in embodiments. In some embodiments, the sensor environment can provide information to a user via display on augmented reality glasses, a cell phone screen, or other display. This information can include highlighting regions of a display shelf which contain one or more products which are not fresh or will not be fresh on a desired date of use, providing a popup message indicating that the product is not fresh or is not sufficiently fresh (which can include more detail such as a freshness rating), and can provide information that it will not be most fresh on the date of the desired use for the product, but will be most fresh on a different date.

After 220, method 200 can return to 208 to perform additional product testing until one or more suitable products are determined. Additional user feedback steps similar to operation 218 can be included in some embodiments, such as after operation 220. Once operation 218 is completed, method 200 ends at 222.

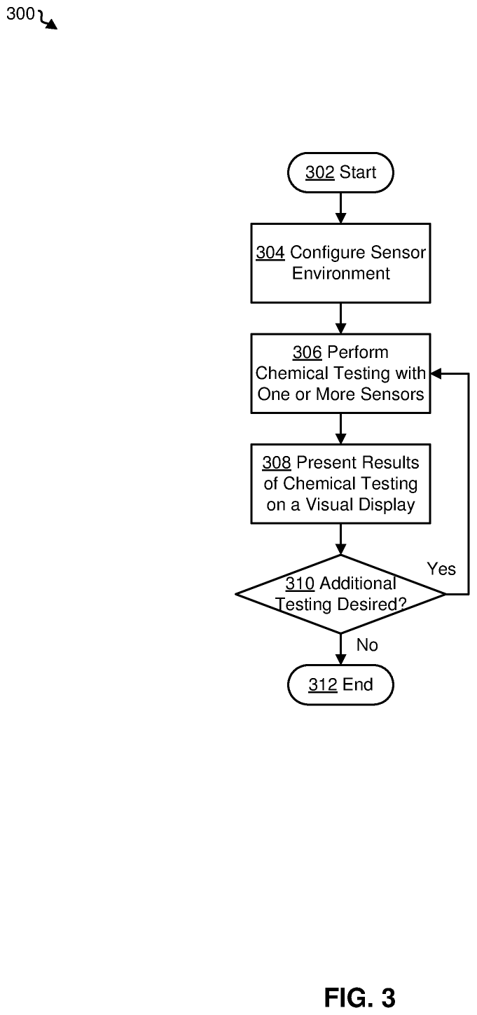

FIG. 3 depicts an example method for chemical detection and visual display using a sensor environment, in accordance with embodiments of the present disclosure. Method 300 can include more or fewer operations than those depicted. Method 300 can include operations in different orders than those depicted. In some embodiments, method 300 can be performed by or performed using a sensor environment (such as sensor environment 100 depicted in FIG. 1) and/or by a computer system (such as computer system 400 depicted in FIG. 4).

From start 302, method 300 proceeds to 304 to configure the sensor environment. Configuration of the sensor environment will vary in embodiments. In some embodiments, configuration of the sensor environment will include turning on augmented reality glasses and/or activating a chemical detection application or setting for such glasses, cell phone, or other device. Configuration of the sensor environment can include connecting and/or activating one or more sensors including augmented reality glasses, a tactile sensor, an olfactory sensor, a bacterial sensor, and/or any additional types of sensors as applicable. Various types of sensors may be useful for certain applications and may be unnecessary for other applications. For example, detecting a trace of a chemical left behind in gaseous form may utilize an olfactory sensor, whereas detecting a most concentrated area of a chemical may utilize a visual sensor such as augmented reality glasses or other camera input. Configuration of the sensor environment may also include activating a computer connected to one or more of the sensors. Configuration of the sensor environment at 304 can also include selecting a mode of operation for the sensor environment in embodiments where more than one mode exists, such as, but not limited to, a food freshness mode, a chemical freshness mode, and/or a chemical sensing mode.

At 306, a user of the sensor environment performs chemical testing with one or more chemical sensors. Using the example from above of a hospital employee seeking the source of a cleaning agent, the hospital employee can perform chemical testing with one or more sensors in the effort to locate the chemical. This can include for example, viewing an area with augmented reality glasses or capturing video of an area, and such viewing can include detection by the glasses or video camera of wavelengths of light which are invisible to the human eye. This can also include using an olfactory sensor or other sensor for detection of gaseous chemicals.

At 308, results of the chemical testing are presented on a visual display. The nature of the presentation can vary in embodiments depending on the visual display involved. In some embodiments, a visual display can be presented in augmented reality glasses such that the visual display is overlaid onto the real environment. The results can be presented to indicate areas where chemicals have been detected, such as by highlighting, marking, or otherwise placing emphasis on a location. The results can also be presented with areas of relative emphasis relating to one or more aspects of the results of the chemical testing. For example, relative concentration of a chemical detected in an area can be marked by using light highlighting on an area with a low concentration, by marking with dark highlighting an area with high concentration, and using gradations between these areas for intermediate concentrations. In another embodiment, the visual display can present results of chemical testing based on relative safety levels of chemicals detected, such as by marking hazardous chemicals in a red color or using a hazard icon, while marking harmless chemicals with a yellow color or using an icon to represent safety. Many forms of display can be used in various embodiments, including charts, graphs, pictorial representations, text-based descriptions, and more.

At 310, a user can determine whether additional testing is desired. Continuing with the example of the hospital employee from above, after testing an initial area (or a single point in an initial area), they can determine that they have not yet located the source of the smell and proceed to collect a variety of samples by returning to operation 306 and performing chemical testing with one or more sensors again. Additional performances of operation 306 can result in updating the visual display by presenting the new or updated results at operation 308. For example, by moving one or more of the sensors involved and performing testing in a three-dimensional area, results for each point tested can be presented in a three-dimensional visual display using augmented reality.

Once additional testing is determined not to be desired at 310, such as when the example hospital employee locates the chemical source, method 300 ends at 312.

Referring now to FIG. 4, illustrated is a block diagram of a computer system 400, in accordance with some embodiments of the present disclosure. In some embodiments, computer system 400 performs operations in accordance with FIGS. 2 and/or 3 as described above. In some embodiments, computer system 400 can be consistent with sensor environment 100 of FIG. 1 or a component thereof, such as data analyzer 112. The computer system 400 can include one or more processors 405 (also referred to herein as CPUs 405), an I/O device interface 410 which can be coupled to one or more I/O devices 412, a network interface 415, an interconnect (e.g., BUS) 420, a memory 430, and a storage 440.

In some embodiments, each CPU 405 can retrieve and execute programming instructions stored in the memory 430 or storage 440. The interconnect 420 can be used to move data, such as programming instructions, between the CPUs 405, I/O device interface 410, network interface 415, memory 430, and storage 440. The interconnect 420 can be implemented using one or more busses. Memory 430 is generally included to be representative of a random access memory (e.g., static random access memory (SRAM), dynamic random access memory (DRAM), or Flash).

In some embodiments, the memory 430 can be in the form of modules (e.g., dual in-line memory modules). The storage 440 is generally included to be representative of a non-volatile memory, such as a hard disk drive, solid state device (SSD), removable memory cards, optical storage, or flash memory devices. In an alternative embodiment, the storage 440 can be replaced by storage area-network (SAN) devices, the cloud, or other devices connected to the computer system 400 via the I/O devices 412 or a network 450 via the network interface 415.

The CPUs 405 can be a single CPU, multiple CPUs, a single CPU having multiple processing cores, or multiple CPUs with one or more of them having multiple processing cores in various embodiments. In some embodiments, a processor 405 can be a digital signal processor (DSP). The CPUs 405 can additionally include one or more memory buffers or caches (not depicted) that provide temporary storage of instructions and data for the CPUs 405. The CPUs 405 can be comprised of one or more circuits configured to perform one or more methods consistent with embodiments of the present disclosure.

The memory 430 of computer system 400 includes sensor control instructions 432 and data analyzer 434. Sensor control instructions 432 can be an application or compilation of computer instructions for controlling one or more sensors attached or otherwise connected to computer system 400. Sensor control instructions can include instructions for receiving information from one or more sensors and/or instructions for sending information to one or more sensors. Instructions for the one or more sensors can include configuration settings including pressure to apply for a tactile sensor, gases to detect for an olfactory sensor, types of bacteria to monitor for a bacterial sensor, or various other types of configuration settings. Instructions for the one or more sensors can also include instructions which manipulate a sensor, such as a robot hand or probing instrument that is part of a sensor.

Data analyzer 434 can be the same as or substantially similar to data analyzer 112 of FIG. 1 and perform the functions described above. Data analyzer 434 can be computer instructions and/or a software application to be run by computer system 400 for analyzing the data received by one or more sensors relating to the freshness of one or more food or other products. Data analyzer 434 can perform this analysis using user preferences 442 and/or reference data 444 described below.

Storage 440 contains user preferences 442 and reference data 444. User preferences 442 can be data in any format which relates to the preferences of one or more users of computer system 400, customers of a user of computer system 400, or individuals otherwise relate to the food or other products to be analyzed with computer system 400. The nature of user preferences 442 can vary in embodiments and examples of types of user preferences are discussed above regarding operation 206 of FIG. 2.

Reference data 444 can be various types of data which data analyzer 434 can use in determining whether food or other products are fresh. Reference data 444 can include information relating to control samples. For example, reference data can include information relating to colors, smells, firmness, bacteria levels, etc. of fresh food or other products and food or other products which are not yet ripe, spoiled, or otherwise not fresh. Reference data 444 can be used by data analyzer 434 in comparing the reference data with data received from one or more sensors to determine whether the food or other product(s) being analyzed are fresh and/or how fresh they are. Reference data 444 can also include information regarding expected future freshness of food or other products. For example, reference data 444 can include information that an avocado of a particular color and firmness will be most ripe in a specified period of time in the future. For another example, reference data 444 can include information about the expected degradation rate of a chemical which can be used in determining when it will no longer match an acceptable purity level. Data analyzer 434 can use this information in reference data 444 in determining whether a food or other product will match a user's intended use for the product. Various other types of data consistent with this disclosure can be included in reference data 444 useful in making product freshness determinations and/or in the performance of methods 200 of FIGS. 2 and 300 of FIG. 3.

In some embodiments as discussed above, the memory 430 stores sensor control instructions 432 and data analyzer 434, and the storage 440 stores user preferences 442 and reference data 444. However, in various embodiments, each of the sensor control instructions 432, data analyzer 434, user preferences 442, and reference data 444 are stored partially in memory 430 and partially in storage 440, or they are stored entirely in memory 430 or entirely in storage 440, or they are accessed over a network 450 via the network interface 415.

In various embodiments, the I/O devices 412 can include an interface capable of presenting information and receiving input. For example, I/O devices 412 can receive input from a user and present information to a user and/or a device interacting with computer system 400. In some embodiments, I/O devices 412 include one or more of augmented reality glasses 102, tactile sensor 104, olfactory sensor 106, bacterial sensor 108, and additional sensor(s) 110 of FIG. 1.

The network 450 can connect (via a physical or wireless connection) the computer system 400 with other networks, and/or one or more devices that interact with the computer system.

Logic modules throughout the computer system 400—including but not limited to the memory 430, the CPUs 405, and the I/O device interface 410—can communicate failures and changes to one or more components to a hypervisor or operating system (not depicted). The hypervisor or the operating system can allocate the various resources available in the computer system 400 and track the location of data in memory 430 and of processes assigned to various CPUs 405. In embodiments that combine or rearrange elements, aspects and capabilities of the logic modules can be combined or redistributed. These variations would be apparent to one skilled in the art.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to explain the principles of the embodiments, the practical application or technical improvement over technologies found in the marketplace, or to enable others of ordinary skill in the art to understand the embodiments disclosed herein.

What is claimed is:

1. A computer-implemented method for determining product characteristics, the method comprising:
receiving one or more user preferences regarding a product characteristic;
performing, using one or more sensors, product testing of a product to gather characteristic data of the product;
determining, using the characteristic data of the product, a measured characteristic value of the product;
comparing the measured characteristic value with an expected characteristic value associated with the one or more user preferences;
determining, based on the comparison, a product suitability value for use by a user; and
outputting, using the product suitability value, an indication of whether the product is suitable for use by the user in accordance with the one or more user preferences.

2. The method of claim 1, wherein the one or more user preferences include an expected date of use of the product.

3. The method of claim 1, wherein the one or more sensors includes augmented reality glasses.

4. The method of claim 3, wherein the indication comprises information displayed to a wearer of the augmented reality glasses overlaid onto images received by the augmented reality glasses.

5. The method of claim 1, wherein the one or more sensors includes a tactile sensor capable of measuring a firmness value for the product and wherein determining the measured characteristic value of the product comprises comparing the firmness value for the product with an expected firmness for the product.

6. The method of claim 1, wherein the one or more sensors includes an olfactory sensor capable of generating a scent profile for the product.

7. The method of claim 6, wherein the scent profile is based on one or more chemicals being emitted by the product.

8. The method of claim 7, wherein determining the product suitability value comprises comparing the scent profile with one or more reference scent profiles and wherein each scent profile corresponds to a different characteristic value.

9. A system for determining product characteristics, the system comprising:
one or more processors;
one or more sensors; and
a memory communicatively coupled to the one or more processors,
wherein the memory comprises instructions which, when executed by the one or more processors, cause the one or more processors to perform a method comprising:
receiving one or more user preferences regarding a product characteristic;
performing, using the one or more sensors, product testing of a product to gather characteristic data of the product;
determining, using the characteristic data of the product, a measured characteristic value of the product;
comparing the measured characteristic value with an expected characteristic value associated with the one or more user preferences;
determining, based on the comparison, a product suitability value for use by a user; and
outputting, based on the product suitability value, an indication whether the product is suitable for use by the user in accordance with the one or more user preferences.

10. The system of claim 9, wherein the one or more user preferences include an expected date of use of the product.

11. The system of claim 9, wherein the one or more sensors includes augmented reality glasses and wherein the indication comprises information displayed to a wearer of the augmented reality glasses overlaid onto images received by the augmented reality glasses.

12. The system of claim 9, wherein the one or more sensors includes a tactile sensor capable of measuring a firmness value for the product and wherein determining the measured characteristic value of the product comprises comparing the firmness value for the product with an expected firmness for the product.

13. The system of claim 9, wherein the one or more sensors includes an olfactory sensor capable of generating a scent profile for the product and wherein the scent profile is based on one or more chemicals being emitted by the product.

14. The system of claim 13, wherein determining the product suitability value comprises comparing the scent profile with one or more reference scent profiles and wherein each scent profile corresponds to a different characteristic value.

15. A computer program product for determining product characteristics, the computer program product comprising a computer readable storage medium having program instructions embodied therewith, wherein the computer readable storage medium is not a transitory signal per se, the program instructions executable by a computer to perform a method comprising:
receiving one or more user preferences regarding a product characteristic;
performing, using one or more sensors, product testing of a product to gather characteristic data of the product;
determining, using the characteristic data of the product, a measured characteristic value of the product;
comparing the measured characteristic value with an expected characteristic value associated with the one or more user preferences;
determining, based on the comparison, a product suitability value for use by a user; and
outputting, using the product suitability value, an indication whether the product is suitable for use by the user in accordance with the one or more user preferences.

16. The computer program product of claim 15, wherein the one or more user preferences include an expected date of use of the product.

17. The computer program product of claim 15, wherein the one or more sensors includes augmented reality glasses and wherein the indication comprises information displayed to a wearer of the augmented reality glasses overlaid onto images received by the augmented reality glasses.

18. The computer program product of claim 15, wherein the one or more sensors includes a tactile sensor capable of measuring a firmness value for the product and wherein determining the measured characteristic value of the product comprises comparing the firmness value for the product with an expected firmness for the product.

19. The computer program product of claim 15, wherein the one or more sensors includes an olfactory sensor capable of generating a scent profile for the product and wherein the scent profile is based on one or more chemicals being emitted by the product.

20. The computer program product of claim 19, wherein determining the product suitability value comprises comparing the scent profile with one or more reference scent profiles and wherein each scent profile corresponds to a different characteristic value.
